(19) United States
(12) Patent Application Publication
(10) Pub. No.: US 2019/0125064 A1
(43) Pub. Date: May 2, 2019

(54) HOLSTER FOR ULTRASOUND IMAGING DEVICE

(71) Applicant: Butterfly Network, Inc., Guilford, CT (US)

(72) Inventors: Matthew de Jonge, New York, NY (US); Stephen Christopher, Philadelphia, PA (US); Jake Eisner, New York, NY (US)

(73) Assignee: Butterfly Network, Inc., Guilford, CT (US)

(21) Appl. No.: 16/172,618

(22) Filed: Oct. 26, 2018

Related U.S. Application Data
(60) Provisional application No. 62/578,252, filed on Oct. 27, 2017.

(51) Int. Cl. A45F 5/02; A61B 8/00

(52) U.S. Cl. CPC A45F 5/02; A45F 2200/0533; A61B 8/4427

(57) ABSTRACT

Described herein is an apparatus and method for attaching an ultrasound imaging device to a user. The apparatus may include a clip configured to clip to a portion of the user's clothing. The apparatus may further include a holding element configured to receive the ultrasound imaging device and hold the ultrasound imaging device in place.

### CROSS-REFERENCE TO RELATED APPLICATIONS

This application claims the benefit under 35 U.S.C. § 119 (e) to U.S. Provisional Application Ser. No. 62/578,252, filed on Oct. 27, 2017, under Attorney Docket No. B1348.70061US00 and entitled “HOLSTER FOR ULTRASOUND IMAGING DEVICE,” which is incorporated herein by reference in its entirety.

### FIELD

Generally, the aspects of the technology described herein relate to ultrasound systems. Some aspects relate to a holster for an ultrasound imaging device configured to hold an ultrasound imaging device on a user.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue (e.g., by using an ultrasound imaging device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an apparatus for holding an ultrasound imaging device on a user. In some embodiments, the apparatus includes a holster configured to hold an ultrasound imaging device. In some embodiments, the holster includes a clip configured to clip to a portion of the user's clothing. In some embodiments, the holster includes one or more retainers configured to hold the ultrasound imaging device in the holster.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows a holster for holding an ultrasound imaging device according to embodiments disclosed herein;

FIG. 2 shows an example view of the holster of FIG. 1;

FIG. 3 illustrates an ultrasound imaging device being inserted into the holster of FIG. 1;

FIG. 4 shows an example view of the holster of FIG. 1;

FIG. 5 shows an example view of the holster of FIG. 1 holding an ultrasound imaging device, with a strap of the holster shown removed;

FIG. 6 shows another example view of the holster of FIG. 1 holding an ultrasound imaging device, with a strap of the holster shown removed;

FIG. 7 shows a holster for holding an ultrasound imaging device according to other embodiments disclosed herein;

FIG. 8 shows an example view of the ultrasound holster of FIG. 7;

FIG. 9 illustrates an ultrasound imaging device being inserted into the holster of FIG. 7, with a clip of the holster shown removed;

FIG. 10 shows another example view of the ultrasound holster of FIG. 7, with a clip of the holster shown removed;

FIG. 11 shows another example view of the ultrasound holster of FIG. 7, with a clip of the holster shown removed;

FIGS. 12A and 12B show example views of the holster of FIG. 7 holding an ultrasound imaging device; and

FIG. 13 shows an example view of a holster holding a mobile electronic device on a patient's bed according to another embodiment disclosed herein.

### DETAILED DESCRIPTION

Conventional ultrasound systems are large, complex, and expensive systems that are typically only purchased by large medical facilities with significant financial resources. For example, ultrasound systems are typically arranged on a large cart that is setup in an exam room or is wheeled around to a patient's bedside for testing. As is known, the carts typically include a screen for viewing and transducers that are stored in the cart.

Recently, cheaper, portable, and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. 2017/0360397 A1, which is incorporated by reference herein in its entirety. The reduced cost and increased portability of these new ultrasound devices may make them significantly more accessible to the general public than conventional ultrasound devices. For example, in some embodiments, the ultrasound imaging device may be connected to a mobile electronic device, such as a smart phone, which may act as the display for a user performing diagnostic imaging and/or treatment on a patient with the ultrasonic imaging device.

The inventors have recognized that advantages may be realized if the user is able to hold the ultrasound imaging device, also referred to herein as ultrasound probe or probe, on his or her person at all desired times. For example, the user may be able to keep the ultrasound probe with him/her while visiting patients during rounds such that imaging and/or treatment may be performed when needed, without the user having to first reserve, locate, and/or transport an ultrasound cart to the patient.

The inventors have also recognized that advantages may be realized if the user is able to removably attach the ultrasound probe to his or her clothing or to a personal item, such as to a bag. For example, the user may clip the ultrasound probe onto the waist area of the user's pants or skirt, to a pocket, or to a jacket. The inventors further recognized that the ultrasound imaging device would benefit by being removably attachable to a holder that, in turn, may attach the ultrasound probe to the user's clothes. For example, the user may remove the ultrasound probe from the holder attached to his/her jacket for use, and thereafter return the ultrasound probe to the holder once imaging and/or treatment is performed.

The inventors have also recognized that the ultrasound probe may benefit from being protected while being carried around by the user. The inventors have further recognized that the ultrasound probe would benefit from having cable management to maintain the cord when the imaging device is not in use. For example, advantages may be realized if the user may tie up or otherwise secure the ultrasound cable to or around the ultrasound probe, or to or around the holster, when the ultrasound probe is not in use.

Embodiments disclosed herein include a holster configured to hold the ultrasound imaging device, or ultrasound probe, on a user. For example, the holster may be configured to hold or otherwise retain the ultrasound probe in the holster and also to attach the holster to a portion of the user's clothing or other personal item. In one such example, the user may clip the holster to the waist area of pants or a skirt, or to a pocket on the clothes of the user. In some embodiments, the holster may allow the user to carry the ultrasound probe on himself/herself, from place to place, and remove the ultrasound probe from the holster when the user may need to perform imaging and/or treatment.

It should be appreciated that the holster may retain the probe in the holster in numerous ways and that the holster may be held on the user in numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect. For example, in some embodiments, the holster may include one or more retainers, such as a cradle or one or more finger, for retaining the ultrasound probe in the holster. The holster also may include a clip for attaching the holster to a user's clothing or personal item. In some embodiments, the holster may include a protective pocket to protect at least a portion of the probe.

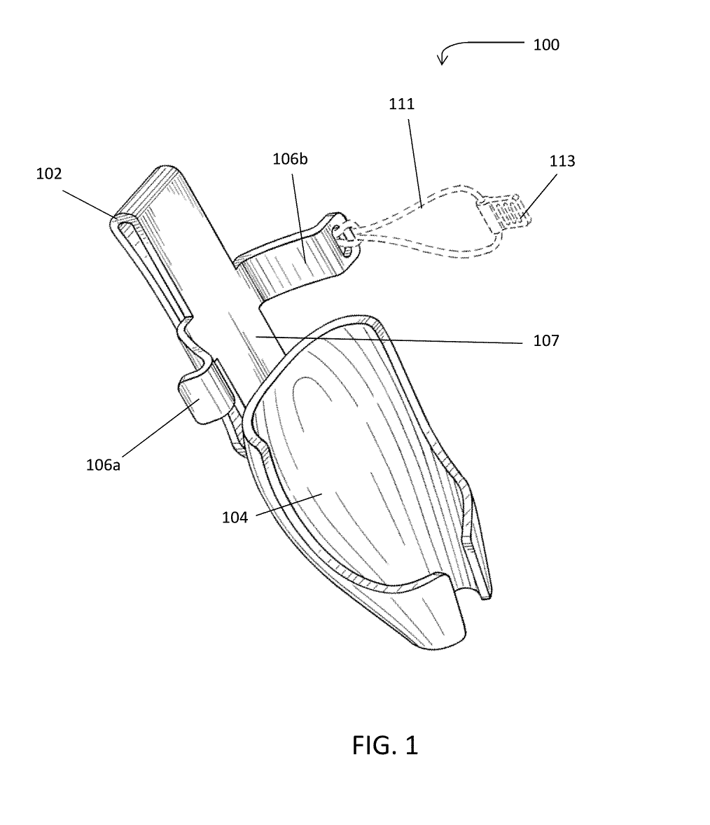

FIG. 1 shows an example holster 100 for retaining an ultrasound imaging device or ultrasound probe on a user. As shown in this figure, the holster 100 may include a clip 102 configured to attach the holster to a portion of the user's clothing, such as to the waist area of pants or a skirt, or to a pocket. The holster also may include one or more retainers 104, 106 for retaining the ultrasound probe in the holster.

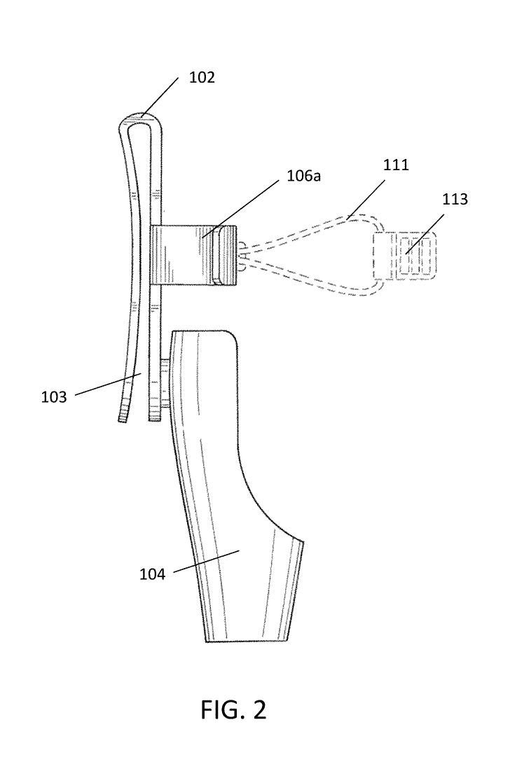

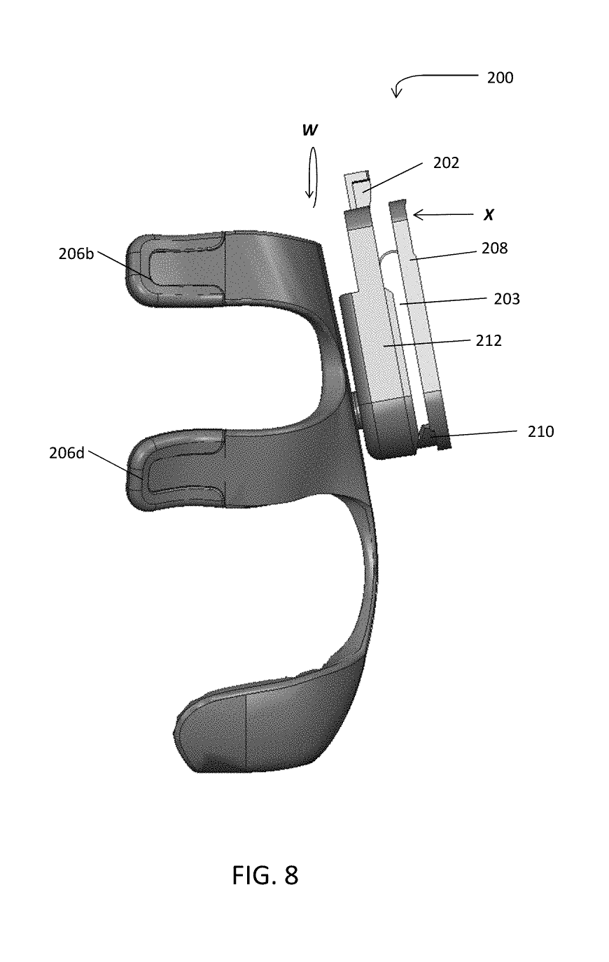

In some embodiments, as shown in FIGS. 1 and 2, the clip 102 includes an opening 103 into which a piece of the user's clothing may be inserted. As will be appreciated, the opening 103 may have any suitable shape and size as this aspect of the disclosure is not limited in this regard. In some embodiments, as shown in FIG. 8, the clip 202 may include an actuator 208 that may be pressed by the user (see arrow X) to access the opening 203 for insertion of a portion of the user's clothing. In some embodiments, the actuator may be biased towards the closed position shown in FIG. 8, such that when the user releases the actuator, the opening may be closed. In some embodiments, the clip may include a protrusion 210 that engages with the actuator to close the opening when the actuator returns to the closed position. As will be appreciated, although the protrusion is shown as being attached to the body of the clip 202 for engagement with the actuator 208, in other embodiments, the protrusion 210 may be attached to the actuator 208 for engagement with the body of the clip. As will be further appreciated, the protrusion may be any shape or size and may engage with a complementary the actuator.

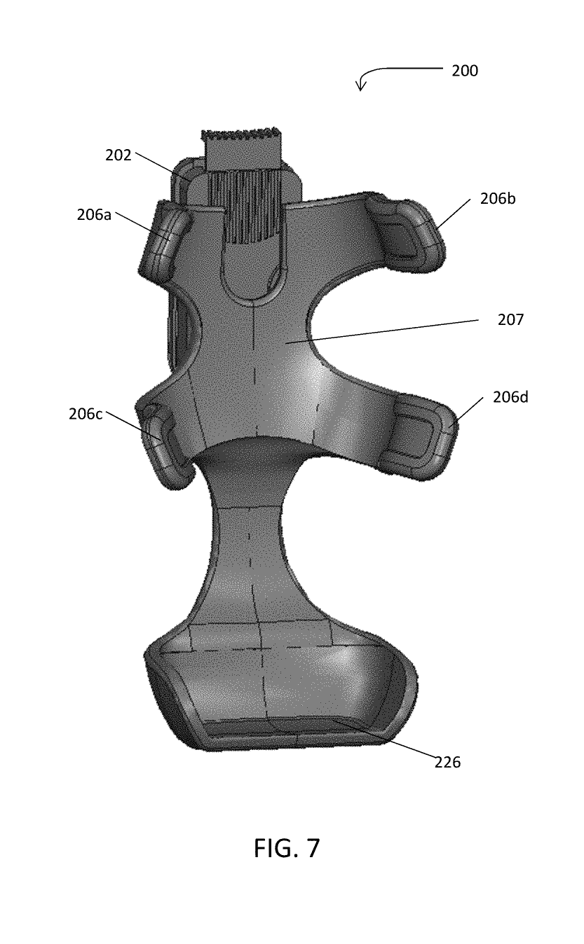

In some embodiments, as shown in FIG. 8, the clip may be rotatably attached to the body of the holster. For example, the clip may be rotatably attached to a rear support member 207 (see FIG. 7). In some embodiments, the holster may be rotated (see arrow W in FIG. 8) to adjust the position of the holster and probe, or the position of an empty holster, relative to the user when the holster is attached to the user's clothing. For example, the user may rotate the holster out of the way when the user is using the probe for imaging and/or treatment.

Although a clip is shown and described for allowing the holster to be held on the user, other arrangements may be used to attach the probe to the user's clothing or to otherwise allow the user to wear the holster. For example, the holster may include a lanyard or other necklace that the user may place around his or her neck to wear the holster. The holster also may be attached to a belt or strap, that the user may secure around his/her waist. As will be further appreciated, the holster need not have a clip. For example, the holster may include only retainers, as will be described, for retaining the ultrasound probe in the holster and protecting the ultrasound probe. In such an example, the user may simply place the holster, with an attached ultrasound probe, in a pocket of his/her jacket or in his/her bag.

In some embodiments, the holster includes one or more retainers for holding the ultrasound probe in the holster. As will be described, the retainers may include a cradle, one or more fingers, other suitable retainers, and combinations thereof. In some embodiments, the retainers are arranged to conform to the shape of the ultrasound probe. For example, the retainers may have a shape that corresponds to a shape of at least a portion of the probe body.

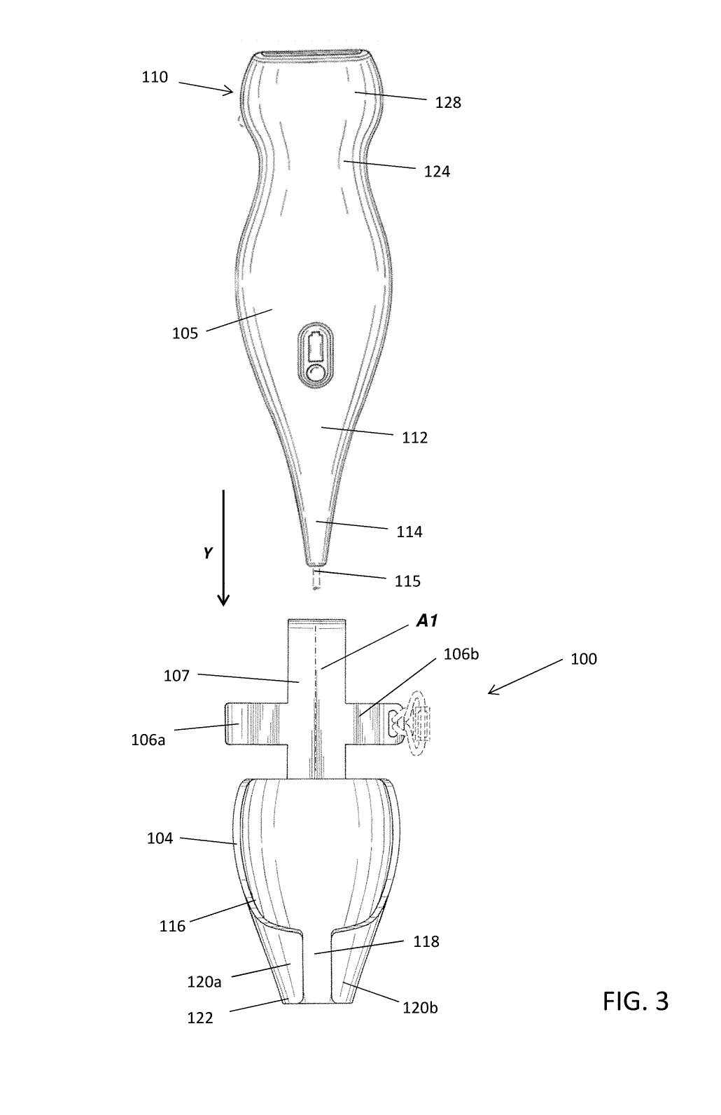

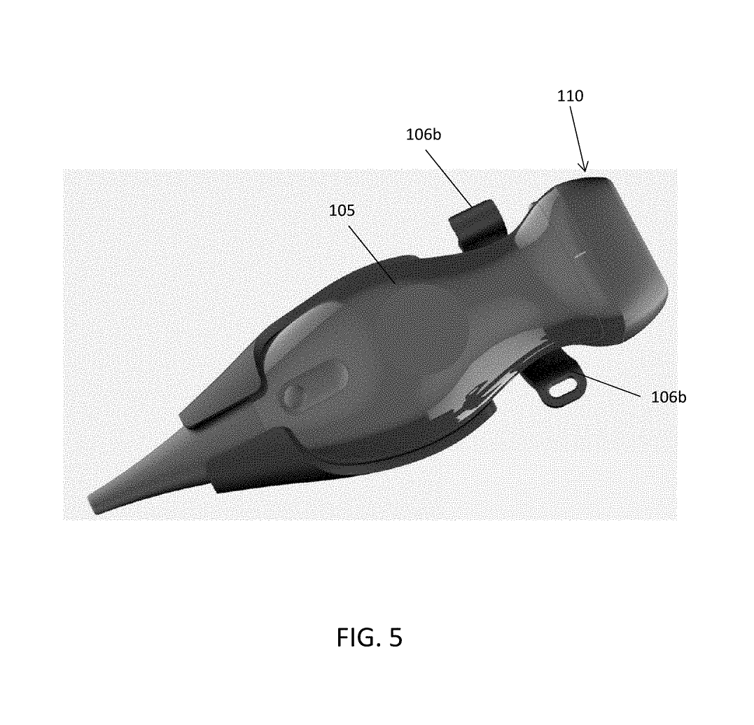

As shown in FIGS. 1 and 2, in some embodiments, the holster include a cradle 104 for holding the ultrasound probe. For example, the cradle 104 may be arranged to hold at least a portion of the body 105 (see FIGS. 3 and 5) of the ultrasound probe 110 when the ultrasound probe inserted into the cradle 104. In some embodiments, the cradle 104 is attached to a rear support member 107. In some embodiments, as shown in FIG. 3, the ultrasound probe 110 may be slid in a direction (see arrow Y) into the cradle. In some embodiments, the direction Y is substantially parallel to a longitudinal axis A1 of the holster. As will be appreciated, the ultrasound probe also may be slid into the cradle at other suitable angles.

In some embodiments, the shape of the cradle may correspond to the shape of the body of the ultrasound probe. For example, as shown in FIG. 3, a first portion 112, such as a base or handle, of the probe body 105 may be tapered, with a diameter of the probe decreasing towards a first end 114 of the probe. In some embodiments, a cable 115 may be attached at the first end 114 of the probe. In some embodiments, the cradle 104 has a body 116 that is also tapered, with a diameter of the cradle, decreasing towards a first end 122 of the cradle. As will be appreciated, the cradle may have other suitable shapes in other embodiments for holding the ultrasound probe in the holster.

In some embodiments, the cradle encircles at least a portion of the ultrasound probe to hold the ultrasound probe in the holster. As will be appreciated in view of at least FIGS. 3 and 5, the cradle need not encircle the entire body of the ultrasound probe. For example, the cradle may encircle only the handle portion of the probe body. As will be further appreciated, only a portion of the cradle may be arranged to encircle the ultrasound probe. For example, as shown in FIG. 5, the front of the cradle is shorter than the back of the cradle and may encircle less of the probe than the back of the cradle.

In some embodiments, the cradle may be configured to expand in diameter to receive the ultrasound probe and then contract in diameter to hold the ultrasound probe in place. For example, the cradle may include first and second flaps 120a, 120b that move away from one another and/or deform in a direction outwardly from the cradle. In such an example, the first and second flaps 120a, 120b may move outwardly and/or deform in a direction away from the longitudinal axis A1 of the holster when the ultrasound probe is being inserted into the holster. In some embodiments, the flaps may snap back once the ultrasound probe is in place in the cradle. As will be appreciated, the cradle need not expand and contract to receive or hold the ultrasound probe. For example, the cradle may be made of a rigid material and may maintain its shape while the user is inserting the ultrasound probe into the cradle.

In some embodiments, the ultrasound probe may be maintained in the cradle via the corresponding shapes of the ultrasound probe and the cradle. For example, as shown in FIG. 3, since both the cradle and the ultrasound probe are tapered, the diameter of the first end 122 of the cradle 104 may be smaller than a diameter of the ultrasound probe above the first end 114 of the ultrasound probe (e.g., towards a middle portion of the probe). In such an example, when the probe is inserted into the cradle and the holster is attached to the user's clothing, the probe may not slide out of the opening at the first end 122 of the cradle. Instead, the cradle may encircle a portion of the probe having a diameter equal to or greater than a diameter of the cradle, retaining the probe in the holster. As will be appreciated, in such embodiments, the force of gravity acting on the probe may act to urge the ultrasound probe towards the first end 122 of the cradle when the probe is inserted in the cradle.

In some embodiments, as shown in FIG. 5, a portion of the probe may extend outwardly beyond the cradle when the ultrasound probe is held in the cradle. For example, the first end (e.g., the base of the probe) and the cable may extend outwardly beyond the first end 112 of the cradle.

In some embodiments, as shown in FIGS. 1-12, the holster may include one or more fingers configured to hold the ultrasound probe in the holster. In some embodiments, as shown in FIGS. 1-5, first and second fingers 106a, 106b may be arranged to hold a neck portion 124 of the probe body 105. As shown in this view, the fingers may be used in embodiments in which the holster also includes a cradle 104. As will be appreciated, in other embodiments, the holster may include only a cradle.

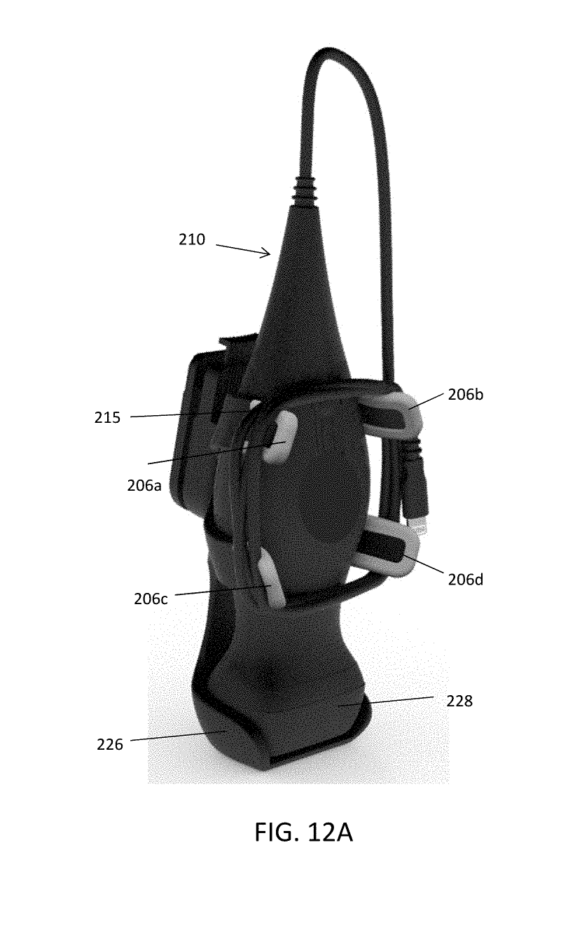

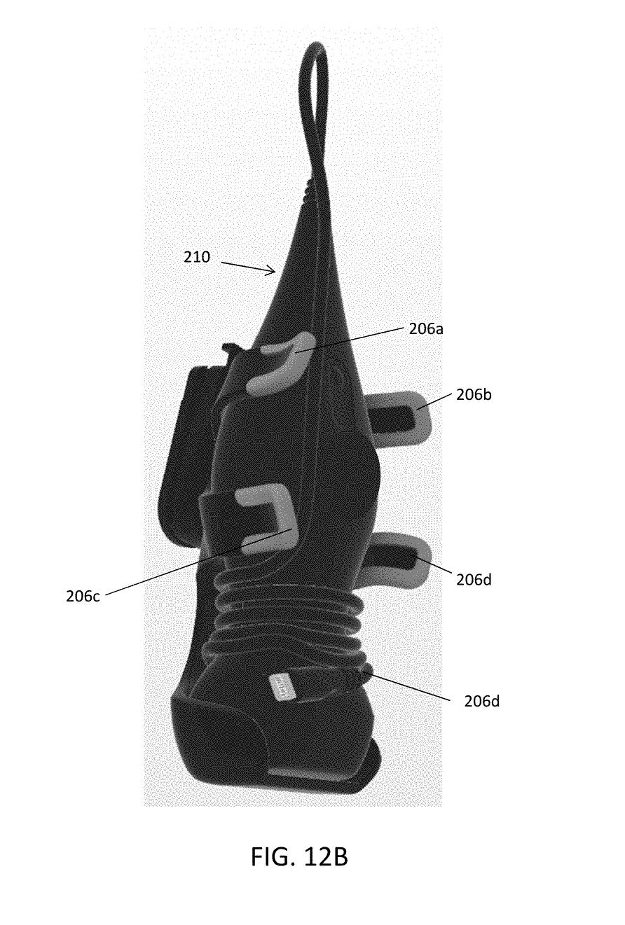

In some embodiments, as shown in FIGS. 7-12B, the holster 200 may include only fingers to retain the ultrasound probe in the holster. For example, the holster 200 may include four fingers 206a-206d to hold the ultrasound probe 210 in the holder. In some embodiments, as shown in FIGS. 12A and 12B, each finger may be arranged to wrap around at least a portion of the probe body. For example, each finger may be arranged to wrap around a portion of a side of the probe body.

For example, the fingers may be positioned around a middle portion of the probe body. In some embodiments, the middle portion of the body is bulbous in shape. As will be appreciated, although fingers are shown as being positioned around a middle portion of the probe body when the probe body is in place, the fingers may be positioned in other suitable locations. For example, fingers may be placed near a neck portion and/or near a handle portion. In some embodiments, by positioning the fingers around a middle portion of the probe, the fingers may prevent the probe from rotating in the holster.

As will be appreciated, although the holster is shown as having four fingers in these embodiments, in other embodiments, the holster may have two or more fingers. As will be further appreciated, the fingers may have any suitable shape and size. For example, the fingers may be substantially rectangular, circular, square, triangular, oval, other polygonal shape, or other suitable shape. In some embodiments, the shape and size of the fingers may be the same for all of the fingers on the holster. In other embodiments, the shape and size of the fingers may vary from finger to finger.

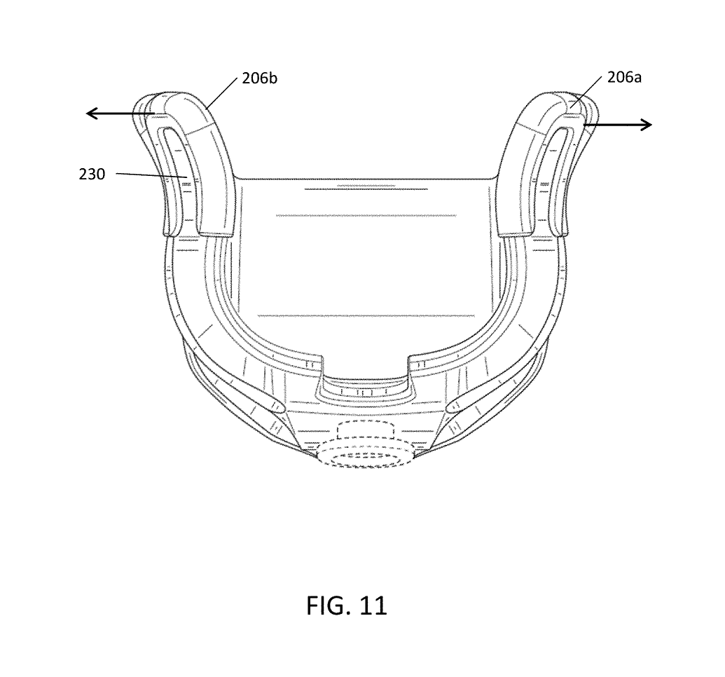

In some embodiments, as shown in FIG. 11, each pair of fingers, along with the back support, may form substantially U-shape. As will be appreciated, the fingers and back support may form other suitable shapes for wrapping around the robe. In some embodiments, the ends of each fingers may curve outwardly from the U-shape.

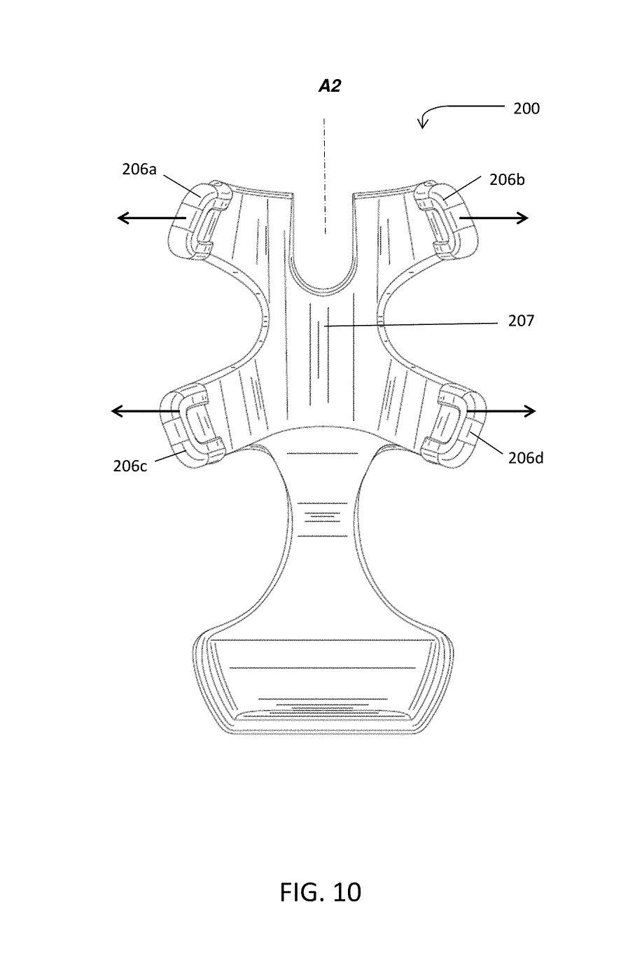

As shown in FIG. 1, in some embodiments, the fingers 106 extend substantially perpendicular to the longitudinal axis A1 of the holster. In other embodiments, as shown in FIG. 10, the fingers 206 may extend at an angle relative to the longitudinal axis A2 of the holster. For example, the fingers may be angled between 0 and 180 degrees relative to the longitudinal axis.

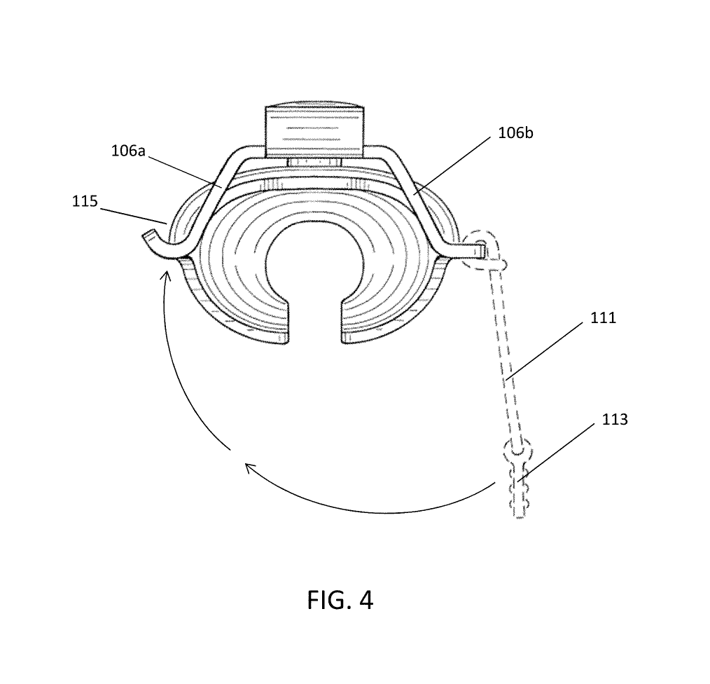

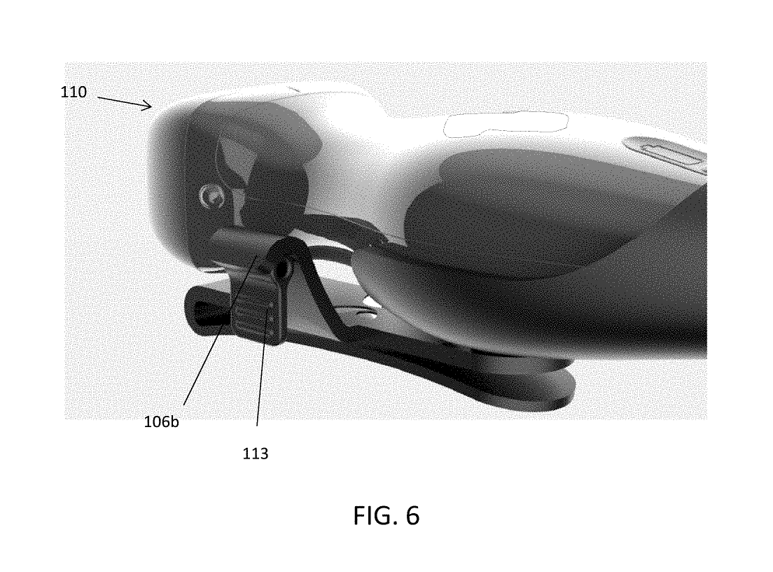

In some embodiments, as shown in FIGS. 1 and 4, the holster may include a strap configured to extend between a pair of fingers 106a, 106b to hold at least a portion of the probe (e.g., a neck portion) to the holster. As shown in these figures, in some embodiments, a first end of the strap may be attached to one of the fingers 106b, with the second end of the strap being removably attachable to the second finger 106a (see FIG. 4). As will be appreciated, the second end of the strap may be attached to the second finger 106a via any suitable method. In some embodiments, as shown in FIG. 1, the second end of the strap 111 may include a fastener 113 that engages with the second finger 106a. For example, as shown in FIG. 6, the outward curve of the finger 106a may create a recess 115 into which the a portion of the fastener 113 may be received. As will be appreciated, to remove the probe from the holster, the user may remove the fastener 113 from the recess formed in the finger 106a and move the strap away from the neck of the probe.

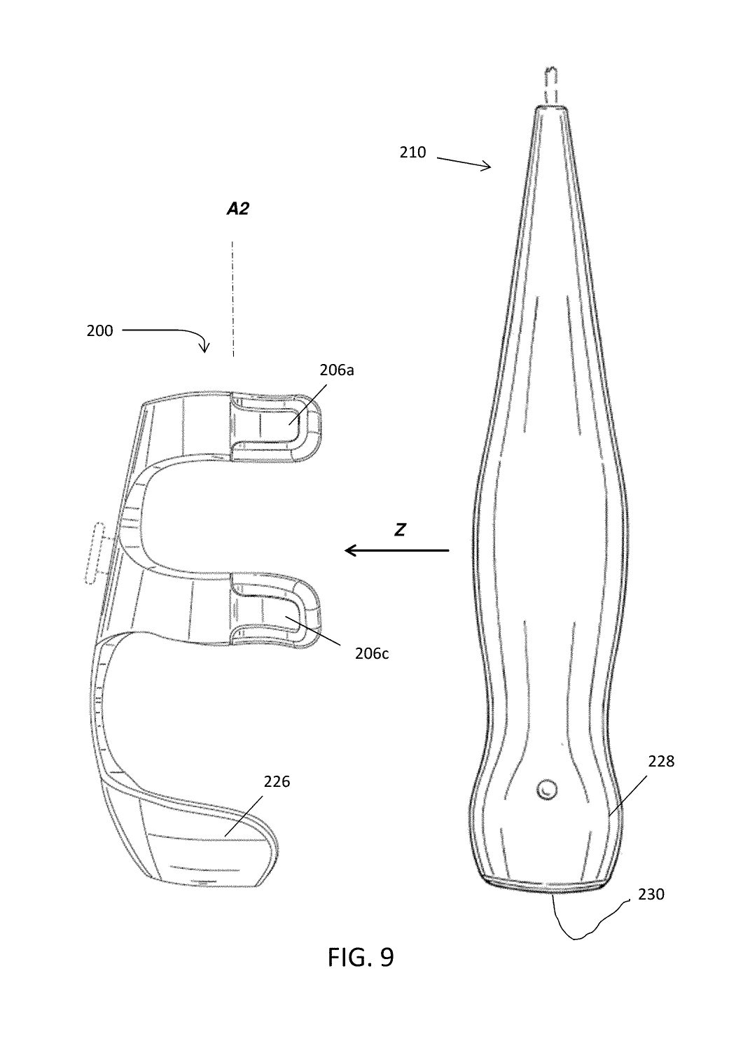

As shown in FIGS. 1-5, in some embodiments, the probe may be inserted in the holster such that the head of the probe is facing upwardly, with the base of the probe in the cradle. In other embodiments, the holster may be arranged such that the probe may be inserted with the head of the probe facing downwardly in the holster, such as that shown in FIGS. 9 and 12A-12B.

In embodiments in which the head of the probe is downwardly facing in the holster, the holster may include a protective pocket 226 arranged to cover and/or encircle at least a portion of the head 228 of the ultrasound probe. In some embodiments, the protective pocket covers at least a top face 230 of the probe (see FIGS. 9 and 12A-12B). As will be appreciated, the top face of the probe may include the portion of the probe that is placed against a portion of the patient's body to perform imaging and/or treatment. In some embodiments, the protective pocket also may extend around one or more sides of the head of the probe. For example, as shown in FIG. 12A, the protective pocket extends around the rear and left and right sides of the head of the probe.

In some embodiments, when the ultrasound probe is held in the holster, the top face of the head of the probe is spaced from the protective pocket. For example, when the probe is held in the holster, the top face may be positioned a distance from the bottom wall of the protective pocket. In such embodiments, the side walls of the protective pocket may contact the sides of the probe head, although the side walls of the protective pocket also may be spaced from the sides of the probe head.

In some embodiments, as shown in FIG. 9, the probe 210 may be moved in a direction Z to insert the probe into the holster. In some embodiments, the direction Z is substantially perpendicular to a longitudinal axis A2 of the holster. In some embodiments, the probe also may inserted in a first direction to move the head of the probe into the protective pocket 226 and then a second direction to engage the ultrasound probe with the fingers and secure the ultrasound probe in the holster. In some embodiments, the first direction and the second direction are different directions. For example, the first direction may be about 45 degrees while the section direction is substantially perpendicular (e.g., about 90 degrees) to the longitudinal axis. As will be appreciated, the ultrasound probe may be moved in other suitable direction to align the probe in the holster and to secure the probe in the holster.

In some embodiments, as shown in FIG. 10, the fingers 206a-206d are arranged to deform and/or deflect outwardly when the probe is being inserted into the holster. For example, the fingers may move outwardly and away from the holster when the probe is being inserted between the fingers. In some embodiments, only a portion of each finger is arranged to move outwardly when the probe is being inserted between the fingers. In some embodiments, the fingers may move outwardly relative to the longitudinal axis of the holster.

In some embodiments, the fingers are arranged to snap back into their original position, such as that shown in FIG. 10, once the ultrasound probe is in place on the holster. In such embodiments the probe may be positioned behind at least a portion of the fingers. For example, as shown in FIGS. 12A and 12B, the probe is located behind the curved end portions of the fingers when the probe is secured in the holster. In such embodiments, the probe may rest against the rear support 207.

In some embodiments, the fingers may be formed of a flexible material. As will be appreciated in view of the above, the fingers may be formed of a material that retains its shape. Such a material may allow the figures to deflect and snap back into a relaxed or starting position after being deflected.

The ultrasound imaging device 110, 210 may be a wired or wireless imaging device. In embodiments in which the ultrasound imaging device 110, 210 is wired, the holster 100, 200 may include an opening, extension, or other configuration for allowing the wire/cable to fit into the holster and/or to wrap around the holster. In this manner, the wire/cable may be suitably contained. For example, as shown in FIG. 1, in some embodiments, the holster may include a slit 118 in a front portion of the cradle 104 for passing through the ultrasound probe cable 115 when the probe is inserted into the cradle. As will be appreciated in view of at least FIG. 7, in embodiments in which the holster includes fingers 206a-206d, the ultrasound probe cable may pass freely in between the fingers when the ultrasound probe is inserted into the holster.

In some embodiments, the holster may allow for the ultrasound probe cord to be secured or otherwise maintained while the probe is held to the holster. In some embodiments, as shown in FIG. 12B, the cord may be wrapped around a neck of the ultrasound probe. As will be appreciated, the cord may be wrapped around the neck of the ultrasound probe prior to inserting the probe in the holster. In some embodiments, such as that shown in FIG. 5, the cord may be wrapped around the first end or base of the ultrasound probe after the ultrasound probe is inserted into the holder. The cord also may be secured in between the fingers 106a, 106b, and the strap 111 of the holster 100 (see FIG. 1).

In another embodiment, as shown in FIG. 12A, the cord may be wrapped around the fingers 206a-206b on the holster. As shown in this figure, the cord may be wrapped around an outwardly-facing side of each finger. In some embodiments, as shown in FIGS. 7 and 10, an end of each finger may be curved such that a retaining surface or recess 230 is formed for receiving the cord being wrapped around the fingers. As will be appreciated, in such embodiments, the distal end of each curved finger may serve to trap the cord in the recess/surface 230 when the cord is wrapped around the fingers. In some embodiments, wrapping the cord around the fingers also may assist in holding the ultrasound probe to the holster.

In some embodiment the end of the cord may include a clip, snap, or other faster which may be attached to another portion of the cord (e.g., to hold the cord being wrapped around the fingers and/or probe). As will be appreciated, the clip may include a channel into which the portion of the cord may be inserted. In such embodiments, the shape of the channel may correspond to the shape of the cord.

It should be appreciated that while two or more retainers (e.g., fingers and a cradle) are shown for holding the ultrasound probe to the holster, in some embodiments, the holster may include one, two, three, four, or more retainers. For example, the holster may include only a cradle to hold the ultrasound probe in some embodiments or may include six fingers. As will be further appreciated, although the holster is shown as having a cradle and fingers for holding the ultrasound probe to the holster, other suitable retaining mechanisms may be used to hold the probe in the holster. For example, in some embodiments, the probe may include a fastener that engages with a corresponding fastener on the holster. The probe also may include a protrusion that is received in a corresponding opening on the holster. The probe may further include a snap, clip, or hook that engages with a corresponding snap, clip, or loop on the holster. As will be appreciated, the holster and probe also may have other suitable arrangements in other embodiments.

It should be appreciated from the foregoing, that a holster for an ultrasound probe may be provided. In some embodiments, the holster may include a fastening mechanism to hold the ultrasound probe. The holster may be couplable to a user, for example mounting to a user's clothing. The holster may optionally include a fastening mechanism to contain a cord or cable of the ultrasound probe.

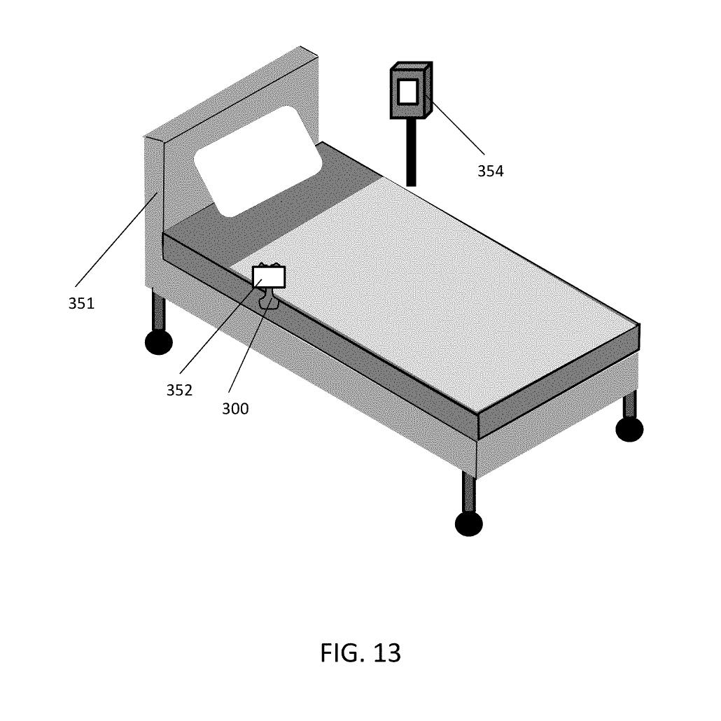

In some embodiments, as shown in FIG. 13, the holster may include one or more retainers arranged to hold a portable electronic device, such as a smart phone, when the user is performing imaging and/or treatment on a patient. In some embodiments, the fingers used to hold the probe in the holster may be used to hold the portable electronic device. In other embodiments, the holster may have other suitable retainers (e.g., a second set of fingers) to hold the portable electronic device. As shown in FIG. 13, the clip (not visible) of the holster may be used to attach the holster to a patient's bed 351, such as by clipping the holster onto a blanket or sheet. The portable electronic device 352, in turn, may be attached to the holster 300 for viewing by the user. As will be appreciated, the holster may be attached to other suitable surfaces, such as to a pole of an intravenous dispensing machine. As will be further appreciated, the holster also may include a handle that the user may grasp to view an attached portable electronic device while performing imaging and/or treatment on a patient.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus for holding an ultrasound imaging device on a user, the apparatus comprising;
a holster configured to hold an ultrasound imaging device.

2. The apparatus of claim 1, wherein the holster includes a clip configured to clip the holster to a portion of a user's clothing.

3. The apparatus of claim 2, wherein the clip includes an opening into which the portion of the user's clothing is insertable.

4. The apparatus of claim 2, wherein the clip is removably attachable to the holster.

5. The apparatus of claim 2, wherein the clip is rotatable relative to the holster.

6. The apparatus of claim 1, wherein the holster includes one or more retainers configured to hold the ultrasound imaging device to the holster.

7. The apparatus of claim 6, wherein the one or more retainers includes a cradle.

8. The apparatus of claim 7, wherein the cradle includes an opening into which a bottom portion of the ultrasound imaging device is insertable.

9. The apparatus of claim 8, wherein the cradle includes a diameter that decreases in size from a first end of the cradle towards a second end of the cradle.

10. The apparatus of claim 7, wherein the cradle is configured to encircle at least a portion of the ultrasound imaging device.

11. The apparatus of claim 6, wherein the one or more retainers include one or more fingers configured to hold the ultrasound probe in the holster.

12. The apparatus of claim 11, wherein the one or more fingers include first and second fingers.

13. The apparatus of claim 12, wherein the first and second fingers extend substantially perpendicular to a longitudinal axis of the holster.

14. The apparatus of claim 12, wherein the first and second fingers are positioned at an angle relative to a longitudinal axis of the holster.

15. The apparatus of claim 12, wherein the first and second fingers are configured to deflect outwardly when the ultrasound imaging device is being inserted into the holster.

16. The apparatus of claim of claim 15, wherein the first and second fingers are configured to snap back into a non-deflected position after the ultrasound imaging device is inserted into the holster.

17. The apparatus of claim 12, wherein the first and second fingers move outwardly relative to the holster when the ultrasound imaging device is being inserted into the holster.

18. The apparatus of claim 12, wherein a distal end of each of the first and second fingers curves outwardly.

19. The apparatus of claim 12, wherein the first and second fingers form a “U” shape with a rear support of the holster.

20. The apparatus of claim 12, further comprising third and fourth fingers, wherein the first, second, third, and fourth fingers are arranged to hold the ultrasound imaging device to the holster.

21. The apparatus of claim 20, wherein the first, second, third, and fourth fingers are configured to wrap around at least a portion of the ultrasound imaging device.

22. The apparatus of claim 12, further comprising a strap that extends between the first and second fingers.

23. The apparatus of claim 22, wherein a first end of the strap is removably attachable to at least one of the first and second fingers.

24. The apparatus of claim 1, wherein the holster includes a protective pocket configured to receive at least a portion of a head of the ultrasound imaging device.

25. The apparatus of claim 1, in combination with an ultrasound imaging device.

26. The combination of claim 25, wherein the ultrasound imaging device is movable in a direction substantially parallel to a longitudinal axis of the holster when the ultrasound imaging device is inserted into the holster.

27. The combination of claim 25, wherein the ultrasound imaging device is movable in a direction substantially perpendicular to a longitudinal axis of the holster when the ultrasound imaging device is inserted into the holster.
